(19) United States
(12) Patent Application Publication
(10) Pub. No.: US 2024/0090828 A1
(43) Pub. Date: Mar. 21, 2024

(54) WAKEFULNESS-LEVEL TINNITUS THERAPY

(71) Applicant: Cochlear Limited, Macquarie University, NSW (AU)

(72) Inventor: Kelly Assouly, Mechelen (BE)

(21) Appl. No.: 18/546,496

(22) PCT Filed: Feb. 2, 2022
(86) PCT No.: PCT/IB2022/050917

§ 371 Date: Aug. 15, 2023

Related U.S. Application Data
(60) Provisional application No. 63/151,846, filed on Feb. 22, 2021.

(51) Int. Cl. A61B 5/00; A61B 5/369; A61N 1/36

(52) U.S. Cl. CPC A61B 5/4809; A61B 5/369; A61B 5/4836; A61N 1/361; A61N 1/36139

(57) ABSTRACT

Presented herein are techniques to managing tinnitus by delivering tinnitus therapy signals to a recipient during time periods in which tinnitus may be most noticeable. The techniques presented herein optimize the tinnitus therapy signals for the recipient by taking into account the recipients state or level of “wakefulness” or “arousal” (e.g., the recipients arousal state). That is, the techniques presented herein manage a recipients tinnitus by adjusting one or more attributes/parameters of the tinnitus therapy signals (e.g., amplitude, frequency, rate, modulation, type, etc.) based on, or in a manner that is dependent upon, the person's wakefulness/arousal state.

### BACKGROUND

### Field of the Invention

The present invention relates generally to the delivery of tinnitus therapy to a recipient.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: capturing one or more wakefulness-state signals with one or more sensors associated with an auditory prosthesis; determining, based on the one or more wakefulness-state signals, a level of wakefulness of a recipient of the auditory prosthesis; and generating tinnitus therapy signals for delivery to the recipient, wherein one or more attributes of the tinnitus therapy signals are set based on the level of wakefulness of the recipient.

In another aspect, a system is provided. The system comprises: at least one sensor configured to capture one or more signals associated with a recipient; a stimulation unit configured to generate tinnitus therapy signals for delivery to the recipient; and a processing unit configured to: monitor, based on the one or more signals, a current arousal state of the recipient, and dynamically adjust, over a period of time, one or more attributes of the tinnitus therapy signals based on the current arousal level of the recipient.

In another aspect, one or more non-transitory computer readable storage media are provided. The one or more non-transitory computer readable storage media comprise instructions that, when executed by a processor, cause the processor to: monitor a level of wakefulness of a recipient; and generate tinnitus therapy control signals based on the level of wakefulness of a recipient.

In another aspect, an implantable medical device system is provided. The implantable medical device system comprises: a plurality of sensors configured to capture one or more wakefulness-state signals associated with a recipient, wherein at least one of the plurality of sensors is an implantable sensor; one or more processors configured to monitor output signals from the plurality of sensors and to determine a current level of wakefulness of the recipient and to generate tinnitus therapy control signals based on the current level of wakefulness of the recipient;
a stimulation unit configured to generate electrical stimulation signals from the tinnitus therapy control signals; and one or more implantable electrodes configured to deliver the electrical stimulation signals to the recipient, wherein the plurality of sensors comprise one or more of an electroencephalogram (EEG) sensor, an electromyography (EMG) sensor, a photoplethysmography (PPG) sensor, a sound sensor, a body noise sensor, a movement sensor, a heart rate sensor, a blood pressure sensor, or a temperature sensor.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a schematic block diagram of a tinnitus therapy system configured to implement aspects of the techniques presented herein;

FIG. 3 is a graph illustrating dynamic adjustments to tinnitus therapy signals, in accordance with certain embodiments presented herein;

FIG. 4 is another graph illustrating dynamic adjustments to tinnitus therapy signals, in accordance with certain embodiments presented herein; and

FIG. 5 is a flowchart of an example method, in accordance with embodiments presented herein.

### DETAILED DESCRIPTION

Tinnitus is the perception of noise or “ringing” in the ears which currently affects an estimated 30 million people in the United States alone. Tinnitus is a common artefact of hearing loss, but can also be a symptom of other underlying conditions, such as ear injuries, circulatory system disorders, etc. Although tinnitus affects can range from mild to severe, almost one-quarter of those with tinnitus describe their tinnitus as disabling or nearly disabling.

Tinnitus has a particularly high prevalence in hearing-impaired and cochlear implant recipients, but a majority of cochlear implant recipients experience tinnitus reduction after cochlear implant activation. Although this particular population of cochlear implant recipients may not suffer from tinnitus when the cochlear implant is activated/on (e.g., delivering electrical stimulation to evoke hearing percepts), these recipients can be still experience tinnitus when the cochlear implant is switched off and/or idle (e.g., in quiet environments). Most often, this situation occurs at nighttime when the cochlear implant recipient is attempting to go to sleep, where his/her cochlear implant is deactivated (e.g., switched off and/or in an idle state such that the cochlear implant is generally not delivering signals in a manner to evoke hearing percepts) and the perception of tinnitus sound is highly noticeable. This tinnitus awareness, in turn, causes difficulties in falling asleep.

Presented herein are techniques to manage tinnitus by delivering tinnitus therapy signals (e.g., subthreshold electrical stimulation signals, suprathreshold electrical stimulation signals, mechanical stimulation, acoustic stimulation (sounds), etc.) to a recipient during time periods in which tinnitus may be most noticeable. The techniques presented herein optimize the tinnitus therapy signals for the recipient by taking into account the recipient's state or level of “wakefulness” or “arousal” (e.g., the recipient's arousal state) in order to maintain or manage a desired arousal state. That is, the techniques presented herein manage a recipient's tinnitus by dynamically adjusting (e.g., in real-time) one or more attributes/parameters of the tinnitus therapy signals (e.g., amplitude, frequency, rate, modulation, signal type, etc.) based on, or in a manner that is dependent upon, the person's wakefulness/arousal state. In this context, the techniques presented herein provide a recipient with a variable amount of tinnitus therapy that moderates the tinnitus symptoms so as to enable the recipient to more easily fall asleep or return to sleep. The proposed techniques bypass the awareness of the recipient and prevent tinnitus from reaching a perceptual level that is likely to catch the attention of the recipient, thereby addressing sleep disorders based on tinnitus awareness.

Aspects of the techniques presented herein titrate stimulation delivered to a recipient in order to achieve a balance between effective-enough tinnitus suppression and minimizing sleep-preventing arousal. In particular, the present inventor has recognized the significance that, if a tinnitus sufferer wakes for a short time during the night in a drowsy state, she needs to keep a low level of wakefulness (arousal) so she can return to sleep, despite her returning tinnitus (as a result of waking up), which has a tendency to increase arousal. As such, the techniques presented herein deliver tinnitus therapy signals (tinnitus therapy stimulation signals) in a manner that takes into account the recipient's arousal state (level of wakefulness) and, in certain aspects, her concurrent activity. In the example of waking up during the night, the system can be titrated to minimize overall arousal and generally assist the recipient in returning to sleep. For instance, motion/movement sensors may detect slow or swaying movements that suggest a drowsy or semi-conscious night-time state. In response, an inaudible or barely audible tinnitus stimulation may be initiated, which balances the need for tinnitus treatment with the need to stay at a low level of arousal. Upon waking in the morning, a more regular stimulation can be delivered.

Merely for ease of description, the techniques presented herein are primarily described with reference to a specific implantable medical device system, namely a cochlear implant system. However, it is to be appreciated that the techniques presented herein may also be partially or fully implemented by other types of devices, including implantable medical devices, computing devices, consumer electronic devices, etc. For example, the techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc. The techniques presented herein may also be partially or fully implemented by consumer devices, such as tablet computers, mobile phones, wearable devices, etc.

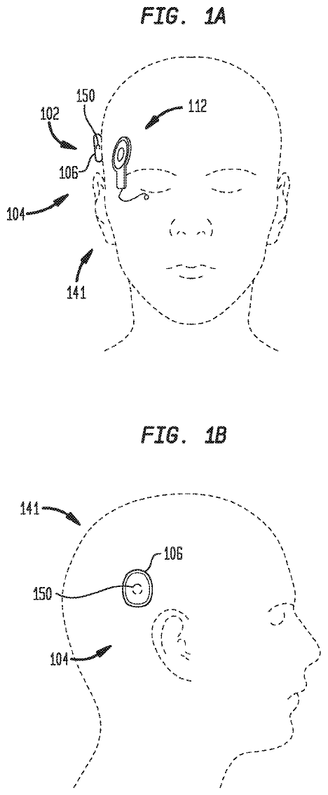

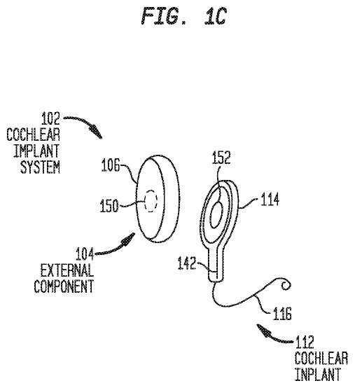

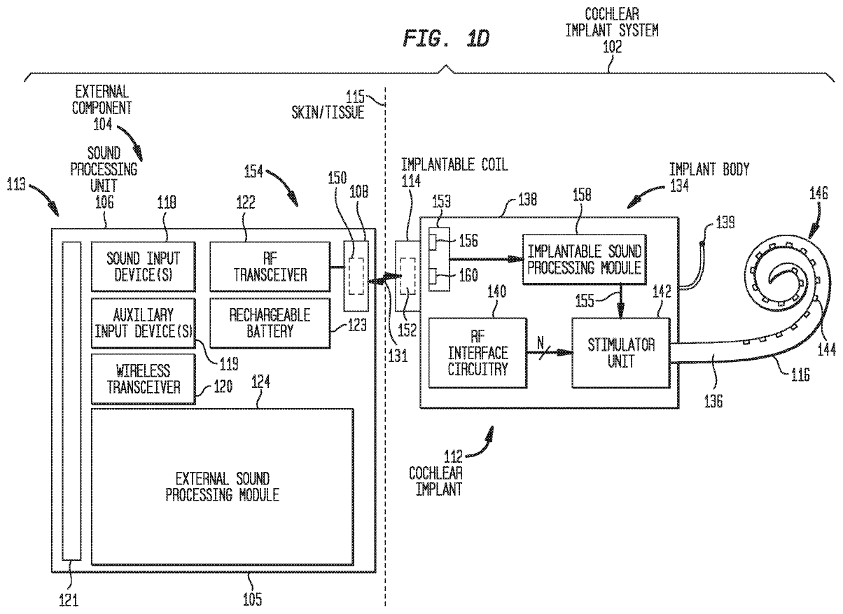

FIGS. 1A-1D illustrates an example cochlear implant system 102 configured to implement certain embodiments of the techniques presented herein. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 141 of a recipient, while FIG. 1B is a schematic drawing of the external component 104 worn on the head 141 of the recipient. FIG. 1C is another schematic view of the cochlear implant system 102, while FIG. 1D illustrates further details of the cochlear implant system 102. For ease of description, FIGS. 1A-1D will generally be described together.

As noted, cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an internal coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 105 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

Referring first to the external hearing mode, FIGS. 1A-1D illustrate that the OTE sound processing unit 106 comprises one or more input devices 113 that are configured to receive input signals (e.g., sound or data signals). The one or more input devices 113 include one or more sound input devices 118 (e.g., one or more external microphones, audio input ports, telecoils, etc.), one or more auxiliary input devices 119 (e.g., audio ports, such as a Direct Audio Input (DAI), data ports, such as a Universal Serial Bus (USB) port, cable port, etc.), and a wireless transmitter/receiver (transceiver) 120. However, it is to be appreciated that one or more input devices 113 may include additional types of input devices and/or less input devices (e.g., the wireless short range radio transceiver 120 and/or one or more auxiliary input devices 119 could be omitted).

The OTE sound processing unit 106 also comprises the external coil 108, a charging coil 121, a closely-coupled transmitter/receiver (RF transceiver) 122, sometimes referred to as or radio-frequency (RF) transceiver 122, at least one rechargeable battery 123, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the transceiver 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless RF link 131 formed between the external coil 108 with the implantable coil 114. In certain examples, the closely-coupled wireless link 131 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices 113) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 153 and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 153 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 153) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 155 that are provided to the stimulator unit 142. The stimulator unit 142 is configured to utilize the output signals 155 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 153 in generating stimulation signals for delivery to the recipient.

As noted above, the cochlear implant 112 comprises implantable sound sensors 153. In certain embodiments, the implantable sound sensors 153 comprise at least two sensors 156 and 160, where at least one of the sensors is designed to be more sensitive to bone-transmitted vibrations than it is to acoustic (air-borne) sound waves. In the illustrative embodiment of FIG. 1D, the implantable sensor 156 is an implantable “sound” sensor/transducer that is primarily configured to detect/receive external acoustic sounds (e.g., an implantable microphone), while the implantable sensor 160 is a “vibration” sensor that is primarily configured to detect/receive internal vibration signals, including body noises (e.g., another implantable microphone or an accelerometer). These sensors can take a variety of different forms, such as another implantable microphone, an accelerometer, etc. However, for ease of description, embodiments presented herein will be primarily described with reference to the use of an implantable microphone as the sound sensor and an accelerometer as the vibration sensor. The increased sensitivity of the accelerometer to vibration signals (e.g., body noises) may be due to, for example, the structure of the accelerometer relative to the microphone, the implanted position of the accelerometer relative to the microphone, etc. For example, in certain embodiments, the accelerometer and the microphone are structurally similar but they are placed in different locations which accounts for the vibration/body noise sensitivity difference. Again, it is to be appreciated that these specific implementations are non-limiting and that embodiments of the present invention may be used with different types of implantable sound sensors

The implantable microphone 156 and the accelerometer 160 can each be disposed in, or electrically connected to, the implant body 134. In operation, the implantable microphone 156 and the accelerometer 160 each detect input signals and convert the detected input signals into electrical signals. The input signals detected by the implantable microphone 156 and the accelerometer 160 can each include external acoustic sounds and/or vibration signals, including body noises.

In accordance with embodiments presented herein, the cochlear implant system 102 (e.g., the external sound processing module 124, the implantable sound processing module 158, an external device, etc.), is configured to perform the wakefulness-based tinnitus therapy techniques presented herein. That is, the cochlear implant system 102 is configured to determine a level of “wakefulness” (wakefulness or arousal level) of a recipient of the cochlear implant system 102 prosthesis and is configured to generate tinnitus therapy signals based on the level of wakefulness of the recipient. That is, the cochlear implant system 102 is configured to dynamically control, set, or adjust attributes/parameters of the tinnitus therapy signals by taking into account the recipient's state or level of “wakefulness” or “arousal.” As used herein, the recipient's level of “wakefulness” or “arousal” refers to a brain state and state of consciousness (state of arousal) in which an individual is conscious and engages in coherent cognitive and behavioral responses to the external world (e.g., a state in which there is a conscious monitoring of the environment and in which there is potential for efficient responsiveness to external stimuli or threats). Being awake (e.g., some level of wakefulness) is the opposite of the state of being asleep in which most external inputs to the brain are excluded from neural processing (e.g., state of sleep in which there is reduced responsiveness to environmental stimuli).

Wakefulness is produced by a complex interaction between multiple neurotransmitter systems arising in the brainstem and ascending through the midbrain, hypothalamus, thalamus and basal forebrain, which is not present when asleep. In general, the longer the brain has been awake, the greater the synchronous firing rates of cerebral cortex neurons (e.g., the higher the level of wakefulness). After sustained periods of sleep, both the speed and synchronicity of the neurons firing are shown to decrease. As such, and as noted above, being “asleep” is the opposite of being “awake” (i.e., an individual who is asleep has no wakefulness level). An individual's sleep cycle can be broken into two main types of sleep: nonrapid eye movement (NREM) and rapid eye movement (REM) stages, where NREM stages should typically make up 75 percent of the sleep time.

As noted above, the techniques presented herein determine a recipient's level of wakefulness or level of arousal, sometimes referred to herein as the recipient's “wakefulness level” and then delivers tinnitus therapy signals to the recipient based on the wakefulness level. That is, one or more attributes of the tinnitus therapy signals are dynamically adjusted based on the wakefulness level. As such, in accordance with embodiments presented herein, as the recipient's wakefulness level changes, the tinnitus therapy signals that are generated and delivered to the recipient also change (i.e., are dynamically adjusted in a corresponding manner).

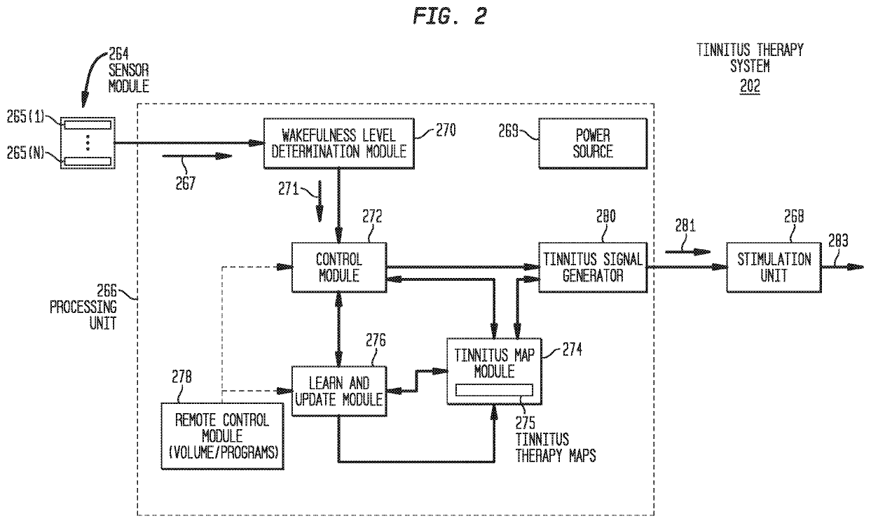

FIGS. 1A-1D illustrate that the wakefulness-based tinnitus therapy techniques presented herein can be performed by one or more components of the cochlear implant system 102, such as the external sound processing module 124, the implantable sound processing module 158, an external device, etc. However, it is also to be appreciated that the wakefulness-based tinnitus therapy techniques can be performed by a variety of other types of devices and that the embodiments of FIGS. 1A-1D are merely illustrative. FIG. 2 is a functional block diagram illustrating an example tinnitus therapy system 202 configured to implement the wakefulness-based tinnitus therapy techniques presented herein. It is to be appreciated that the functional blocks illustrated in FIG. 2 can be implemented across one or more different devices or components that can be implanted in, or external to, the body of a recipient. The tinnitus therapy system 202 can comprise or be a component of, for example, a medical device system, a computing device, a consumer electronic device, etc. Moreover, as used herein, the term “recipient” is used to generic refer to any user of a tinnitus therapy system, such as tinnitus therapy system 202, who suffers from tinnitus. The recipient can also suffer from hearing impairments other than tinnitus.

As shown, the tinnitus therapy system 202 comprises a sensor unit 264, a processing unit 266, and a stimulation unit 268. Again, the sensor unit 264, the processing unit 266, and the stimulation unit 268 can each be implemented across one or more different devices and, as such, the specific configuration shown in FIG. 2 is merely illustrative.

The sensor module 264 comprises a plurality of sensors 265(1)-265(N) that are each configured to capture signals representing the “wakefulness-state” of the recipient (e.g., sensors configured to capture data useable to determine the recipient's wakefulness or arousal level). The signals captured by the sensors 265(1)-265(N) are referred to herein “wakefulness-state data” or “wakefulness-state signals” and can take a number of different forms and can be captured by a number of different sensors. For example, the sensors 265(1)-265(N) can comprise sound sensors (e.g., microphones), movement sensors (e.g., accelerometers), body noise sensors, medical sensors, such as electroencephalogram (EEG) sensors (e.g., one or more external or implantable electrodes and one or more associated recording amplifiers configured to record/measure electrical activity in the recipient's brain), electromyography (EMG) sensors or other muscle or eye movement detector (e.g., one or more external or implantable electrodes and one or more associated recording amplifiers configured to record/measure muscle response or electrical activity in response to a nerve's stimulation of the muscle), photoplethysmography (PPG) sensor (e.g., sensors configured to optically detect volumetric changes in blood in peripheral circulation), electro-oculogram (EOG) sensors, polysomnographic sensors, heart rate sensors, temperature sensors, etc. (e.g., recording heart rate, blood pressure, temperature, etc.). It is to be appreciated that this list of sensors configured to capture wakefulness-state data to assist in measuring a degree and/or kind of current arousal level is merely illustrative and that other sensors can be used in alternative embodiments.

In FIG. 2, the wakefulness-state signals captured by the sensors 265(1)-265(N) are converted into electrical input signals (if not already in an electrical form), which are represented in FIG. 2 by arrow 267. As shown, the wakefulness-state signals 267 (electrical input signals) are processed at the processing unit 266 and used to generate tinnitus therapy signals 283 for delivery to the recipient. To this end, the processing unit 266 comprises a power source (e.g., one or more batteries) 269, a wakefulness-level determination module 270, a control module 272, a tinnitus map module 274, learn and update module 276, a remote control module 278, and tinnitus signal generator 280. It is to be appreciated that the functional arrangement shown in FIG. 2 is merely illustrative and does not require or imply any specific structural arrangements. The various functional modules shown in FIG. 2 can be implemented in any combination of hardware, software, firmware, etc., and one or more of the modules could be omitted in different embodiments.

As shown, the wakefulness-state signals 267 are analyzed by the wakefulness-level determination module 270 to determine the level of wakefulness (wakefulness or arousal level, also defined as awareness or attention status) of the recipient. For example, in one illustrative arrangement, the wakefulness level is determined based on EEG activity and muscle tone, where low-voltage fast EEG activity and high muscle tone indicate a high level of wakefulness. In contrast, NREM sleep can be characterized by high-amplitude low-frequency EEG and decreased muscle tone, while and REM sleep has low-voltage fast EEG activity coupled with a complete loss of muscle tone (REM muscle atonia) and characteristic rapid eye movements which contrast with the slow rolling eye movements observed during NREM.

The wakefulness-level determination module 270 generates wakefulness-level data 271, which includes/indicates the level of wakefulness of the recipient at a given point in time (current arousal level), as determined from the wakefulness-state signals 267 (i.e., the results of the analysis of the wakefulness-state signals 267 detected by the sensors 265(1)-265(N)). As noted above, in addition to the wakefulness-level determination module 270, the processing unit 266 also functionally includes a control module 272. The control module 272 is configured to use the wakefulness-level data 271 (e.g., current wakefulness or arousal level) to select, set, determine, or otherwise adjust a tinnitus therapy for the recipient, as a function of the recipient's level of wakefulness (e.g., determine an appropriate tinnitus therapy for the recipient, given the recipient's current wakefulness level data). Stated differently, the tinnitus therapy that is to be provided to the recipient is specifically determined and adjusted, in real-time, based at least the recipient's level of wakefulness at the current point in time. In certain embodiments, the tinnitus therapy that is to be provided to the recipient is also adjusted based on the recipient's activity level (e.g., amount of movement by the recipient during a given time period).

In accordance with embodiments presented herein, the tinnitus therapy includes the delivery of stimulation signals (stimulation) to the recipient. These stimulation signals, sometimes referred to herein as “tinnitus therapy signals” or “tinnitus relief signals,” are generated by the stimulation unit 268 and are represented in FIG. 2 by arrow 283. The tinnitus therapy signals can have a number of different forms (e.g., electrical stimulation signals, mechanical stimulation signals, acoustic stimulation signals, or combinations thereof) and underlying objectives. For example, in certain embodiments, the tinnitus therapy signals 283 can be masking signals that are configured to mask/cover the recipient's tinnitus symptoms (e.g., expose the recipient to sounds/noises at a loud enough volume that it partially or completely covers the sound of their tinnitus). In other embodiments, the tinnitus therapy signals 283 can be distraction signals that are configured to divert the recipient's attention from the sound of tinnitus. In other embodiments, the tinnitus therapy signals 283 can be habituation signals that are configured to assist the recipient's brain in reclassifying tinnitus as an unimportant sound that should can be consciously ignored. In still other embodiments, the tinnitus therapy signals 283 can be neuromodulation signals that are configured to minimize the neural hyperactivity thought to be the underlying cause of tinnitus. In certain embodiments, the tinnitus therapy signals 283 can be any combination of masking signals, distraction signals, habituation signals, and/or neuromodulation signals.

As noted, in the example of FIG. 2, the tinnitus therapy system 202 includes the simulation unit 268 that is configured to generate the tinnitus therapy signals 283, whether configured for masking, distraction, habituation, and/or neuromodulation purposes. However, shown in FIG. 2 is a tinnitus signal generator 280 that is configured to generate tinnitus therapy control signals 281 that are used to control the stimulation arrangement 268 in a manner determined by the control module 272.

The tinnitus therapy control signals 281 generated by the tinnitus signal generator 280 can dictate a number of different attributes/parameters for the tinnitus therapy signals 283. For example, the tinnitus therapy control signals 281 can be such that the tinnitus therapy signals 283 will be pure tone signals, multi tone signals, broadband noise, narrowband noise, low-pass filtered signals, high-pass filtered signals, band-pass filter signals, predetermined recordings, etc. The tinnitus therapy control signals 281 can also set modulations in the tinnitus therapy signals 283, transitions, etc. It is to be appreciated that these specific parameters are merely illustrative and that the tinnitus therapy signals 283 can have any of a number of different forms.

As described elsewhere herein, the tinnitus therapy signals 283 can be electrical stimulation signals, mechanical stimulation signals, electro-mechanical stimulation signals (e.g., electrical signals and mechanical signals delivered simultaneously or in close temporal proximity to one another), acoustic stimulation signals, electro-acoustic stimulation signals (e.g., electrical signals and acoustic signals delivered simultaneously or in close temporal proximity to one another), etc.

As noted above, the control module 272 is configured to determine the tinnitus therapy based on the wakefulness-level data 271. For example, in certain embodiments, the control module 272 can be configured to dynamically adjust a level (amplitude) of the tinnitus therapy signals 283 based on the wakefulness level of the recipient (e.g., from a level of zero to a max level). In other embodiments, the control module 272 can be configured to adjust a frequency or modulation of the tinnitus therapy signals 283 based on the wakefulness level of the recipient. In still further embodiments, the control module 272 can be configured to adjust the type of tinnitus therapy signals 283 (e.g., select one of, or switch between, masking signals, distraction signals, habituation signals, and/or neuromodulation purposes) 283 based on the wakefulness level of the recipient. In the case that the tinnitus therapy signals 283 are electrical stimulation (current) signals, the control module 272 can be configured to adjust one or more of the current level, pulse rate or pulse width of the tinnitus therapy signals 283.

In the specific example of FIG. 2, the processing unit 266 includes a tinnitus map module 274 that is configured to store a plurality of different tinnitus therapy maps 275. In general, each of the tinnitus therapy maps 275 is a set/collection of parameters that, when selected and used by the control module 272 and/or tinnitus signal generator 280, control the generation of the tinnitus therapy signals (e.g., used to generate tinnitus therapy control signals 281). The parameters can control the sound type (e.g., white noise, wave sounds, rain sounds, etc.), fluctuation or modulation rate, amplitude, sound or masker level settings, on/off, pitch settings transition time settings, etc. In operation, different tinnitus therapy maps 275 can be created (e.g., by the software, an audiologist/clinician, through artificial intelligence, etc.) for different situations (i.e., different combinations of body noise classification(s) and environmental classifications). In operation, there will be maps for different therapies, such as specific maps for masking, specific maps for distraction, specific maps for habituation, specific maps for retraining, etc.

In the example of FIG. 2, the control module 272 is configured to analyze the wakefulness-level data 271 and select one of the tinnitus therapy maps 255 for use in generating the tinnitus therapy signals delivered to the recipient and/or dynamically adjust settings attributes of the tinnitus therapy signals 283. However, it is to be appreciated that the presence of multiple tinnitus maps is merely illustrative and that other embodiments could include one or zero tinnitus maps. For example, the tinnitus map module 274 could be omitted in alternative embodiments and, instead, the control module 272 is configured to determine the tinnitus therapy control signals 281 based on pre-programmed settings and the wakefulness level of the recipient. That is, the specific use of tinnitus therapy maps is merely illustrative and that embodiments presented herein can be implemented without the use of stored tinnitus maps.

In certain examples, selected tinnitus therapy settings can be used to provide tinnitus therapy until the wakefulness-level data 271 changes in manner that causes the control module 272 to select or adjust the tinnitus therapy. Once the tinnitus therapy adjustment is selected for use, the control module 272 could manage the transition between the settings to avoid unintended issues (e.g., annoyance to the recipient). As described further below, the wakefulness-level determination module 270 can determine that the recipient is asleep and the tinnitus therapy can be deactivated (e.g., terminated or reduced to a minimum level in an increment manner) in response to the recipient falling asleep.

For example, initially, the control module 272 is programmed to select a specific tinnitus therapy given specific wakefulness-level data 271 (i.e., programmed to select specific tinnitus therapy settings given a specific level of wakefulness). In certain embodiments, the initial programming of control module 272 can be based on normative data for a population of different recipients. The initial programming of control module 272 to select a specific tinnitus therapy map or can also or alternatively be based on predetermined selection settings that are set/determined for the recipient during a fitting session (e.g., a clinician directed session, a remote care session, etc.). That is, in certain embodiments, the initial programming of control module 272 is based preferences of the recipient, sometimes referred to herein as recipient-specific fitting data.

As noted above, the processing unit 266 also comprises a remote control module 278 and a learn and update module 276. In certain embodiments, the remote control module 278 and the learn and update module 276 are configured to update/adjust, over time, what tinnitus therapy map is selected by the control module 272 based, for example, on recipient preferences.

More specifically, in the case that the tinnitus therapy comprises audible signals, the remote control module 278 is configured to receive recipient requests to change the tinnitus therapy. These recipient setting requests, which can be received from a user interface, or wirelessly from a remote control device, external component, mobile application, etc., indicate the changes that the recipient wants to make some change to the tinnitus therapy (e.g., increase volume, change noise type, select different tinnitus relief type, etc.). The recipient's requested changes can be acted upon by the control module 272 to adjust, in real-time or in the future, the applied tinnitus therapy (i.e., change parameters of the tinnitus therapy signals 283 being delivered to the recipient).

In addition to being acted upon by the control module 272, recipient's requested changes, as well as the wakefulness data, are also provided to the learn and update module 276. Since the learn and update module 276 also has knowledge of the wakefulness level of the recipient (e.g., has access to the wakefulness-state data 271) and has knowledge of what tinnitus relief settings were being utilized (i.e., which tinnitus therapy map 275 was active), the learn and update module 276 is configured to implement an automated learning or adaption process to learn what tinnitus relief settings are optimal for the recipient (e.g., which signals and parameter settings enable the recipient to go to sleep the fastest, which signals and parameter settings are preferred by the recipient, etc.).

As noted above, the tinnitus therapy system 202 is configured to deliver stimulation signals to the recipient in order to remediate her tinnitus. The stimulation signals, referred to herein as tinnitus therapy signals, can be subthreshold signals (e.g., inaudible electrical stimulation signals) or suprathreshold (e.g., audible electrical stimulation signals). As noted, while the tinnitus therapy signals are delivered to the recipient, one or more attributes/parameters of the tinnitus therapy signals (e.g., amplitude) are dynamically adapted/adjusted based on the wakefulness level of the recipient. In certain embodiments, the tinnitus therapy signals can be delivered (and adapted) for a certain amount of time after the system is switched off or idle, to enable the recipient to fall asleep in a silent environment. For example, once the system 202 is turned off, the tinnitus therapy signals are activated, and the amplitude of the stimulation decreases with the wakefulness level of the recipient and stays activated until the wakefulness-level determination module 270 determines that the recipient is asleep.

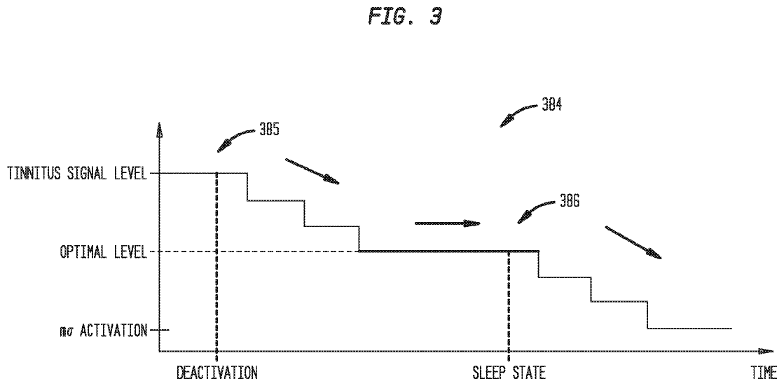

FIG. 3 is a graph 384 illustrating example adjustment to the amplitude of the tinnitus therapy signals (vertical axis) over a period of time (horizontal axis) when a recipient is attempting to initially fall asleep, in accordance with embodiments presented herein. For ease of description, FIG. 3 will be described with reference to tinnitus therapy system 202 of FIG. 2.

In FIG. 3, the tinnitus therapy system 202 is deactivated (e.g., turned off, enters in an idle state, etc.) at point 385 and tinnitus therapy signals are delivered to the recipient at an initial maximum amplitude/level. Thereafter, the amplitude of the tinnitus signals is decreased in manner that corresponds to the recipient's level of wakefulness. At point 386, the tinnitus therapy system 202 (e.g., wakefulness-level determination module 270) determines that the recipient has fallen asleep (e.g., entered the sleep state). Once the recipient has fallen asleep and is no longer awake, the tinnitus therapy system 202 deactivates delivery of the tinnitus therapy signals to the recipient. As shown in FIG. 3, to deactivate delivery of the tinnitus therapy signals to the recipient, the system 202 can incrementally decrease the level of the tinnitus therapy signals to zero (e.g., off) or a minimum level. That is, instead of stopping the therapy abruptly, the system 202 is configured to decrease the level linearly or in a series of discrete steps until the signals are at a level where the recipient is not aware of the activation state.

In FIG. 3, the illustrated “optimal level” can be generalized as the minimum level where tinnitus is relieved/improved/masked. A distinction is made between the minimum level to relieve tinnitus (e.g., the ‘level plateau’ or ‘optimal level’) and the tinnitus signal level which corresponds to the more efficient level when the recipient is fully awake. The optimal level (level plateau) is determined based on a series of test (e.g., with a clinician or done remotely) and corresponds to the minimum value of a specific set of parameters for which the tinnitus is improved. As noted, the optimal level is different from the tinnitus signal level for which tinnitus is improved/suppressed the most efficiently.

Subsequently, if the wakefulness-level determination module 270 determines that the recipient awakens (e.g., transitions from asleep to awake), the control module 272 can reactivate the tinnitus therapy signals automatically until the wakefulness-level determination module 270 determines that the recipient is again asleep and/or the system 202 is activated (e.g., the wakefulness state signals indicate the recipient no longer desires to sleep). The tinnitus therapy signals can be re-activated, for example, based on internal sensors (sound/physiological measurement indicating sleep difficulties) or external sensors (sound/movement or EEG indicating sleep difficulties). The activation can induce an increase of the stimulation until a level plateau (e.g., sub-threshold). Once the wakefulness-level determination module 270 determines that the recipient is again asleep, the control module 272 can again deactivate the tinnitus therapy signals, as described above (e.g., gradually decrease the level of the tinnitus therapy signals to the minimum level).

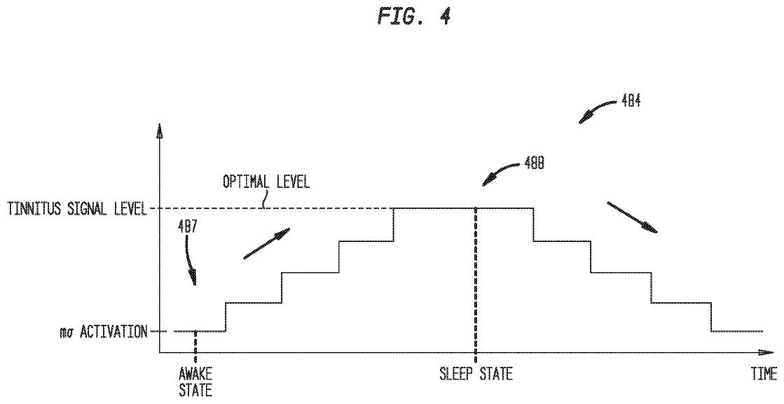

For example, FIG. 4 is a graph 484 illustrating example adjustment to the amplitude of the tinnitus therapy signals (vertical axis) over a period of time (horizontal axis) when a recipient wakes from a sleep state and is attempting to fall back asleep, in accordance with embodiments presented herein. For ease of description, FIG. 4 will also be described with reference to tinnitus therapy system 202 of FIG. 2.

In FIG. 4, the tinnitus therapy system 202 (e.g., wakefulness-level determination module 270) determines that the recipient has awoken from the sleep state. As such, the control module 272 activates the tinnitus therapy signals at a minimum amplitude/level. Thereafter, the level of the tinnitus signals is increased in manner that corresponds to the recipient's level of wakefulness. At point 488, the tinnitus therapy system 202 (e.g., wakefulness-level determination module 270) determines that the recipient has fallen asleep (e.g., entered the sleep state). Once the recipient has again fallen asleep, the level of the tinnitus therapy signals can again be deactivated, as described above (e.g., gradually decrease the level of the tinnitus therapy signals to the minimum level).

In certain embodiments, once the tinnitus therapy signals are activated, the amplitude of the signals will increase/decrease gradually depending on the activation mode. A gradual amplitude increase/decrease over time may be more natural and comfortable for a recipient and can manage arousal levels. The stimulation activation, duration, and modulation can also or alternatively be adjusted automatically by the control module 272 and/or by the recipient via remote control module 278. As noted above, the stimulation needs for each arousal level and context, can be programmed for individualized treatment and management (e.g., in tinnitus therapy maps 275). Other scenarios are similarly contemplated when arousal levels are affected, such as during day napping, chronic insomnia, when affected by medication, or during a meditation session. There is a possibility to extend such stimulation during other disabling situations.

As described elsewhere herein, the tinnitus therapy techniques presented herein can be implemented by stand-alone implantable tinnitus therapy devices, incorporated as part of an auditory prosthesis, such as a cochlear implant, bone conduction device, middle ear auditory prosthesis, direct acoustic stimulator, auditory brain stimulator, etc., implemented by a mobile computing device (e.g., mobile phone), etc. Aspects of the techniques presented herein may be particularly application to recipients that who sleep with a device that is capable of delivering the tinnitus therapy, such as totally implantable cochlear implant or an otherwise sleep-wearable stimulation device. Recipients of interest are those who experiencing tinnitus relief through intracochl ear electrical stimulation. An alternative is to adapt this sleep solution to recipients who experiences tinnitus relief with sound therapy. Thus, the device will provide sound instead inaudible electrical stimulation and will be activated in the same process as described above.

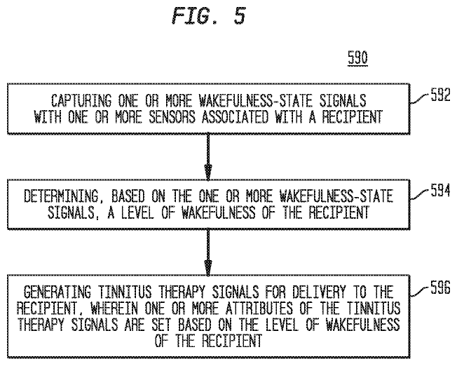

FIG. 5 is a flowchart of an example method 590, in accordance with embodiments presented herein. Method 590 begins at 592 where one or more sensors associated with a recipient (e.g., recipient of an auditory prosthesis, user of a mobile computing device, etc.). As used herein, sensors “associated with” a recipient are sensors configured (e.g., arranged, positioned, etc.) to capture signals/data representing a wakefulness level or arousal state of the recipient. As described above, the one or more sensors can be implanted in, worn by, or positioned in proximity to, the recipient.

Returning to the example of FIG. 5, at 594 one or more processors are configured to determine, based on the one or more wakefulness-state signals, a level of wakefulness of the recipient. At 594, tinnitus therapy signals are generated for delivery to the recipient, where one or more attributes/parameters of the tinnitus therapy signals are set based on the level of wakefulness of the recipient.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method, comprising:
capturing one or more wakefulness-state signals with one or more sensors associated with a device;
determining, based on the one or more wakefulness-state signals, a level of wakefulness of a recipient of the device; and
generating tinnitus therapy signals for delivery to the recipient, wherein one or more attributes of the tinnitus therapy signals are set based on the level of wakefulness of the recipient.

2. (canceled)

3. (canceled)

4. The method of claim 1, wherein capturing the one or more wakefulness-state signals with one or more sensors comprises:
capturing at least one of one or more of sound signals and one or more body noises associated with recipient.

5. The method of claim 1, wherein capturing the one or more wakefulness-state signals with one or more sensors comprises:
recording electrical activity associated with the recipient's brain or recording electrical activity associated with one or more muscles in the body of the recipient.

6. (canceled)

7. The method of claim 1, wherein capturing the one or more wakefulness-state signals with one or more sensors comprises:
recording one or more of a heart rate, blood pressure, or temperature of the recipient.

8. (canceled)

9. (canceled)

10. (canceled)

11. The method of claim 1, wherein generating the tinnitus therapy signals comprises:
dynamically adjusting one or more parameters of the tinnitus therapy signals based on the level of wakefulness of the recipient.

12. The method of claim 11, wherein dynamically adjusting one or more parameters of the tinnitus therapy signals based on the level of wakefulness of the recipient comprises:
dynamically adjusting an amplitude of the tinnitus therapy signals based on the level of wakefulness of the recipient.

13. The method of claim 11, wherein dynamically adjusting one or more parameters of the tinnitus therapy signals based on the level of wakefulness of the recipient comprises:
dynamically setting a modulation of the tinnitus therapy signals based on the level of wakefulness of the recipient.

14. The method of claim 11, wherein the tinnitus therapy signals are electrical stimulation signals, and wherein dynamically adjusting one or more parameters of the tinnitus therapy signals based on the level of wakefulness of the recipient comprises:
dynamically setting at least one of a current level, a pulse rate, or a pulse width of the electrical stimulation signals based on the level of wakefulness of the recipient.

15. The method of claim 1, further comprising:
receiving a user input in response to delivery of the tinnitus therapy signals to the recipient, wherein the user input indicates a recipient adjustment to the tinnitus therapy signals; and
adjusting the tinnitus therapy signals based on the user input.

16. The method of claim 1, further comprising:
determining, based on the one or more wakefulness-state signals, that the recipient has entered a sleep state; and
in response to determining the recipient has entered a sleep state, deactivating the tinnitus therapy signals.

17. The method of claim 16, wherein deactivating the tinnitus therapy signals comprises:
incrementally decreasing an amplitude of the tinnitus therapy signals over a period of time.

18. The method of claim 16, further comprising:
determining, based on the one or more wakefulness-state signals that the recipient has awoken from the sleep state;
in response to determining the recipient has awoken from the sleep state, re-activating the tinnitus therapy signals;
determining the level of wakefulness of the recipient following the sleep state; and
dynamically adjusting one or more parameters of the tinnitus therapy signals based on the level of wakefulness of the recipient following the sleep state.

19. (canceled)

20. A system, comprising:
one or more sensors configured to capture one or more wakefulness-state signals associated with a recipient;
a stimulation unit configured to generate tinnitus therapy signals for delivery to the recipient; and
one or more processors configured to:
monitor, based on the one or more wakefulness-state signals, a current arousal state of the recipient, and
dynamically adjust, over a period of time, one or more attributes of the tinnitus therapy signals based on the current arousal state of the recipient.

21. The system of claim 20, wherein the one or more sensors include at least one electroencephalogram (EEG) sensor, at least one electromyography (EMG) sensor, or at least one photoplethysmography (PPG) sensor.

22. (canceled)

23. (canceled)

24. The system of claim 20, wherein one or more sensors include at least one of a sound sensor, a body noise sensor, or a movement sensor.

25. (canceled)

26. (canceled)

27. The system of claim 20, wherein at least one of the one or more sensors is configured to record one or more of a heart rate, blood pressure, or temperature of the recipient.

28. The system of claim 20, wherein to dynamically adjust one or more attributes of the tinnitus therapy signals based on the current arousal state of the recipient, the one or more processors are configured to:
dynamically adjust an amplitude of the tinnitus therapy signals based on the current arousal state of the recipient.

29. The system of claim 20, wherein to dynamically adjust one or more attributes of the tinnitus therapy signals based on the current arousal state of the recipient, the one or more processors are configured to:
dynamically set a modulation of the tinnitus therapy signals based on the current arousal state of the recipient.

30. The system of claim 20, wherein the tinnitus therapy signals are electrical stimulation signals, and wherein to dynamically adjust one or more attributes of the tinnitus therapy signals based on the current arousal state of the recipient, the one or more processors are configured to:
dynamically adjust at least one of a current level, a pulse rate, or a pulse width of the electrical stimulation signals based on the current arousal state of the recipient.

31. The system of claim 20, wherein the one or more processors are configured to:
determine, based on the one or more wakefulness-state signals, that the recipient has entered a sleep state; and
in response to determining the recipient has entered a sleep state, deactivate the tinnitus therapy signals.

32. The system of claim 31, wherein to deactivate the tinnitus therapy signals, the one or more processors are configured to:
incrementally decrease an amplitude of the tinnitus therapy signals over a period of time.

33. The system of claim 31, wherein the one or more processors are configured to:
determine, based on the one or more wakefulness-state signals that the recipient has awoken from the sleep state;
in response to determining the recipient has awoken from the sleep state, re-activate the tinnitus therapy signals;
determine the current arousal state of the recipient following the sleep state; and
dynamically adjust one or more attributes of the tinnitus therapy signals based on the current arousal state of the recipient following the sleep state.

34. (canceled)

35. The system of claim 20, wherein the system comprises an auditory prosthesis, and wherein the auditory prosthesis is configured to deliver the tinnitus therapy signals to the recipient.

36. (canceled)

37. One or more non-transitory computer readable storage media comprising instructions that, when executed by a processor, cause the processor to:
monitor a current level of wakefulness of a recipient; and
generate tinnitus therapy control signals based on the current level of wakefulness of a recipient.

38. The one or more non-transitory computer readable storage media of claim 37, wherein the instructions operable to monitor the current level of wakefulness of a recipient comprise instructions operable to:
continually receive wakefulness-state signals from one or more sensors associated with the recipient; and
determine the current level of wakefulness of a recipient at a given point in time based on the wakefulness-state signals.

39. The one or more non-transitory computer readable storage media of claim 37, wherein the instructions operable to generate tinnitus therapy control signals based on the current level of wakefulness of a recipient comprise instructions operable to:
dynamically adjust, over a period of time, one or more attributes of the tinnitus therapy control signals based on the current level of wakefulness of the recipient.

40. The one or more non-transitory computer readable storage media of claim 39, wherein the instructions operable to dynamically adjust, over a period of time, one or more attributes of the tinnitus therapy control signals based on the current level of wakefulness of the recipient comprise instructions operable to:
dynamically adjust an amplitude of the tinnitus therapy control signals based on the current level of wakefulness of the recipient.

41. The one or more non-transitory computer readable storage media of claim 39, wherein the instructions operable to dynamically adjust, over a period of time, one or more attributes of the tinnitus therapy control signals based on the current level of wakefulness of the recipient comprise instructions operable to:
dynamically adjust a modulation of the tinnitus therapy signals based on the current level of wakefulness of the recipient.

42. The one or more non-transitory computer readable storage media of claim 39, wherein the tinnitus therapy control signals are useable to generate electrical stimulation signals are electrical stimulation signals, and wherein the instructions operable to dynamically adjust, over a period of time, one or more attributes of the tinnitus therapy control signals based on the current level of wakefulness of the recipient comprise instructions operable to:
dynamically adjust at least one of a current level, a pulse rate, or a pulse width of the electrical stimulation signals based on the current level of wakefulness of the recipient.

43. (canceled)
